(19) United States
(12) Patent Application Publication
(10) Pub. No.: US 2023/0414956 A1
(43) Pub. Date: Dec. 28, 2023

(54) Methods For Treating Diabetes With Alternating Electric Fields

(71) Applicant: NOVOCURE GMBH, Root D4 (CH)

(72) Inventors: Tali Voloshin-Sela, Haifa (IL); Lilach Avigdor, Haifa (IL)

(21) Appl. No.: 18/342,303

(22) Filed: Jun. 27, 2023

Related U.S. Application Data
(60) Provisional application No. 63/355,821, filed on Jun. 27, 2022.

(51) Int. Cl. A61N 1/40

(52) U.S. Cl. CPC A61N 1/40

(57) ABSTRACT

Disclosed are methods of treating that can be performed using alternating electric fields applied in a single direction or multiple directions. Disclosed are method of treating a subject having diabetes comprising applying alternating electric fields to a target site of the subject for a period of time, the alternating electric fields having a frequency and field strength, wherein FGF21 is increased in the subject.

### CROSS-REFERENCE TO RELATED APPLICATIONS

This application claims the benefit of U.S. Provisional Patent Application No. 63/355,821 filed on Jun. 27, 2022, which is incorporated by reference herein in its entirety.

### BACKGROUND

Multiple studies have concluded that FGF21 lowers blood glucose levels and inhibits glucagon secretion. Administration of FGF21 to rodents or non-human primates causes considerable pharmacological benefits on a cluster of obesity-related metabolic complications. Geng et al. The therapeutic potential of FGF21 in metabolic diseases: from bench to clinic. Nature Reviews Endocrinology 16, 654-667 (2020). Additionally, administering recombinant FGF21 is problematic due to pharmacokinetics.

Described herein is a method of activating FGF21 through application of alternating electric fields, thus solving the problem of administering recombinant FGF21 by inducing endocrine secretion of FGF21.

### BRIEF SUMMARY

Disclosed are methods of treating that can be performed using alternating electric fields applied in a single direction or multiple directions.

In some aspects, disclosed are methods of treating a subject having diabetes comprising applying an alternating electric field to a target site of the subject for a period of time, the alternating electric field having a frequency and field strength, wherein FGF21 is increased in the subject.

In some aspects, disclosed are methods of treating a subject comprising identifying a subject with diabetes, applying an alternating electric field to a target site of the subject for a period of time, the alternating electric field having a frequency and field strength, wherein FGF21 is increased in the subject.

In some aspects, disclosed are methods of treating a subject comprising: identifying a subject with diabetes, wherein the subject is also identified as being tumor-free, applying an alternating electric field to a target site of the subject for a period of time, the alternating electric field having a frequency and field strength, wherein FGF21 is increased in the subject.

Disclosed are methods of treating a subject having diabetes comprising applying an alternating electric field to a target site of the subject for a period of time, the alternating electric field having a frequency and field strength, wherein FGF21 is increased in the subject, and further comprising administering a diabetes therapeutic to the subject.

Disclosed are methods of increasing FGF21 in a subject having diabetes comprising: applying an alternating electric field to a target site of the subject for a period of time, the alternating electric field having a frequency and field strength, wherein FGF21 is increased in the subject.

Disclosed are methods of increasing blood glucose uptake in a subject having diabetes comprising: applying an alternating electric field to a target site of the subject for a period of time, the alternating electric field having a frequency and field strength, wherein FGF21 is increased in the subject.

Disclosed are methods of increasing BDNF in a subject having diabetes comprising: applying an alternating electric field to a target site of the subject for a period of time, the alternating electric field having a frequency and field strength, wherein FGF21 and BDNF are increased in the subject.

Additional advantages of the disclosed methods and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed methods and compositions.

In the figures, alternating electric fields are also identified as “TTFields”.

FIG. 1 shows FGF21 expression in alternating electric fields treated rats compared to heat sham treated cells.

FIG. 2 shows an example of cytokine array analyses showing an increase in BDNF expression in alternating electric fields treated cells.

FIG. 3 shows the treatment protocol used for FIG. 2.

FIG. 4 shows that FGF-21 expression is upregulated following 10 days of alternating electric fields application in an ovarian cancer mice model. The cytokine array results are from in vivo experiments as validation for FGF21 upregulation. An in vivo ovarian cancer model experiment design is shown. Female mice were treated with alternating electric fields for 10 days vs. the control sham heat group. Images of spots from the mouse cytokine array membranes of alternating electric fields treated mice and control sham heat mice are shown. The graph shows quantification of FGF21 pixel density from the cytokine array membranes spots.

### DETAILED DESCRIPTION

The disclosed methods and compositions may be understood more readily by reference to the following detailed description of particular embodiments and the Example included therein and to the Figures and their previous and following description.

It is to be understood that the disclosed methods and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a target site” includes a plurality of such target sites, reference to “the subject” is a reference to one or more subjects and equivalents thereof known to those skilled in the art, and so forth.

As used herein, a “target site” is a specific site or location within or present on a subject or patient. For example, a “target site” can refer to, but is not limited to a cell (e.g., a hepatocyte), population of cells, organ, tissue, or a tumor. Thus, the phrase “target cell” can be used to refer to a target site, wherein the target site is a cell. In some aspects, a “target cell” can be a hepatocyte. In some aspects, organs that can be target sites include, but are not limited to, the liver. In some aspects, a cell or population of cells that can be a target site or a target cell include, but are not limited to, a hepatocyte. In some aspects, a “target site” can be a liver.

As used herein, an “alternating electric field” or “alternating electric fields” refers to a very-low-intensity, directional, intermediate-frequency alternating electric field delivered to a subject, a sample obtained from a subject or to a specific location within a subject or patient (e.g., a target site such as a cell). In some aspects, the alternating electric field can be in a single direction or multiple directional. In some aspects, alternating electric fields can be delivered through two pairs of transducer arrays that generate perpendicular fields within the target site. For example, for the Optune™ system (an alternating electric fields delivery system for treatment of glioblastoma multiforme) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

Array placement on a subject can be optimized to increase the intensity in the target site or target cell. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target site or target cell as a function of the positions of the array and, in some aspects, take account for variations in the electrical property distributions within the heads of different patients.

The term “subject” refers to the target of administration, e.g., an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field. For example, the subject of administration can be a subject with diabetes.

By “treat” is meant to administer or apply a therapeutic, such as an alternating electric field, to a subject, such as a human or other mammal (for example, an animal model), that has diabetes or has an increased susceptibility for developing diabetes, in order to prevent or delay a worsening of the effects of the disease or infection, or to partially or fully reverse the effects of diabetes. For example, treating a subject having diabetes can comprise delivering a therapeutic to a cell in the subject.

By “prevent” is meant to minimize or decrease the chance that a subject develops a condition, for example, diabetes.

As used herein, the terms “administering” and “administration” refer to any method of providing a therapeutic to a subject directly or indirectly to a target site. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat diabetes. In further various aspects, a preparation can be administered prophylactically; that is, administered for prevention of diabetes. In an aspect, the skilled person can determine an efficacious dose, an efficacious schedule, or an efficacious route of administration so as to treat a subject. In some aspects, administering comprises exposing or applying. Thus, in some aspects, exposing a target site or subject to alternating electrical fields or applying alternating electrical fields to a target site or subject means administering alternating electrical fields to the target site or subject.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise an alternating electric field. In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes. In other aspects a single pair of electrodes are placed on opposite sides of the subject and the alternating electric field between them is considered unidirectional.

In some aspects, the frequency of the alternating electric field is between 50 kHz and 10 MHz. In some aspects, the frequency of the alternating electric field is between kHz and 1 MHz. In some aspects, the frequency of the alternating electric field is between 100 and 500 kHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 180 and 220 kHz, between 210 and 400 kHz, or between 500 kHz and 1 MHz. In some aspects, the frequency of the alternating electric fields can be electric fields at or about 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be around 300 kHz.

In some aspects, the field strength of the alternating electric fields can be between 0.5 and 10 V/cm RMS. In some aspects, the field strength of the alternating electric fields can be between 0.5 and 4 V/cm RMS. In some aspects, the field strength of the alternating electric fields can be between 1 and 4 V/cm RMS. In some aspects, different field strengths can be used (e.g., between 0.1 and 10 V/cm). In some aspects, the field strength can be 1.75 V/cm RMS. In some embodiments the field strength is at least 1 V/cm RMS. In some aspects, the field strength can be 0.9 V/cm RMS. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the application may last for at least 6 hours, at least 12 hours, at least 24 hours, at least 36 hours, at least 48 hours, or at least 72 hours or more.

The disclosed methods comprise applying one or more alternating electric fields to a cell or to a subject. In some aspects, the alternating electric field is applied to a target site. When applying alternating electric fields to a cell, this can often refer to applying alternating electric fields to a subject comprising a cell. Thus, applying alternating electric fields to a target site (e.g., liver) of a subject results in applying alternating electric fields to a cell (e.g., hepatocyte).

### C. Methods of Treating

Disclosed are methods of treating that can be performed using an alternating electric field applied in a single direction or multiple directions. Thus, in some aspects a unidirectional alternating electric field can be used (e.g., a single pair of electrodes or transducer arrays) and in some aspects, multidirectional alternating electric fields can be used (e.g., two or more pairs of electrodes or transducer arrays).

Disclosed are methods of treating a subject having diabetes comprising applying an alternating electric field to a target site of the subject for a period of time, the alternating electric field having a frequency and field strength, wherein FGF21 is increased in the subject. Thus, in some aspects, the subject has already been diagnosed/identified as having diabetes. In some aspects the alternating electric field is applied in a single direction. In other aspects the alternating electric field is applied in multiple directions.

Disclosed are methods of treating a subject comprising: identifying a subject with diabetes, applying an alternating electric field to a target site of the subject for a period of time, the alternating electric field having a frequency and field strength, wherein FGF21 is increased in the subject. Thus, in some aspects, the method includes first identifying a subject with diabetes, for example diagnosing a patient with diabetes or identifying that the patient has previously been diagnosed with diabetes. In some aspects the alternating electric field is applied in a single direction. In other aspects the alternating electric field is applied in multiple directions.

Disclosed are methods of treating a subject comprising: identifying a subject with diabetes, wherein the subject has a target site that is identified as being tumor-free, applying an alternating electric field to the target site (e.g., liver) of the subject for a period of time, the alternating electric field having a frequency and field strength, wherein FGF21 is increased in the subject. In some aspects, not only does the method include first identifying a subject with diabetes but also identifying that the target site of the subject is tumor-free. In some aspects a target site is identified as being tumor-free if the patient has not been diagnosed as having a tumor in the target site. Thus, in some aspects it is not just about treating a subject with diabetes but also treating a subject with diabetes whose target site (e.g., liver) is tumor-free and/or cancer-free. In some aspects the alternating electric field is applied in a single direction. In other aspects the alternating electric field is applied in multiple directions.

Disclosed are methods of treating a subject comprising: identifying a subject with diabetes, wherein the subject is also identified as being tumor-free, applying an alternating electric field to a target site of the subject for a period of time, the alternating electric field having a frequency and field strength, wherein FGF21 is increased in the subject. In some aspects, not only does the method include first identifying a subject with diabetes but also identifying that the subject is tumor-free. In some aspects a subject is identified as being tumor-free if the subject has not been diagnosed as having a tumor. Thus, in some aspects it is not just about treating a subject with diabetes but also treating a subject with diabetes who is tumor-free and/or cancer-free. In some aspects the alternating electric field is applied in a single direction. In other aspects the alternating electric field is applied in multiple directions.

In some aspects, disclosed are methods of treating a subject comprising identifying a subject with diabetes, applying an alternating electric field to a target site of the subject for a period of time, the alternating electric field having a frequency and field strength, wherein FGF21 is increased in the subject.

In some aspects, disclosed are methods of treating a subject comprising: identifying a subject with diabetes, wherein a target site (e.g., liver) of the subject is identified as being tumor-free, applying an alternating electric field to the target site of the subject for a period of time, the alternating electric field having a frequency and field strength, wherein FGF21 is increased in the subject. In some aspects a target site is identified as being tumor-free if the patient has not been diagnosed as having a tumor in the target site.

In some aspects, disclosed are methods of treating a subject comprising: identifying a subject with diabetes, wherein the subject is also identified as being tumor-free, applying an alternating electric field to a target site of the subject for a period of time, the alternating electric field having a frequency and field strength, wherein FGF21 is increased in the subject. In some aspects a subject is identified as being tumor-free if the subject has not been diagnosed as having a tumor.

In some aspects, the target site is a location on a subject to provide treatment with an alternating electric field. In some aspects, the target site comprises hepatocytes. For example, the target site can be the subject's liver. In some aspects, the target site is the subject's torso. In some aspects, the target site can be any area of the subject comprising organs or cells associated with the diabetes disease process.

In some aspects, the frequency of the alternating electric fields can be any of the frequencies described throughout. For example, in some aspects, the frequency of the alternating electric fields is between 50 kHz and 10 MHz, 100 kHz and 1 MHz, 100 and 500 kHz, 150 and 500 kHz or 500 kHz and 1 MHz. In some aspects, the frequency of the alternating electric fields is about 100, about 130, about 150, about 180, about 200, about 220, about 250, about 300, about 350, about 400, about 450, or about 500 kHz.

In some aspects, the field strength of the alternating electric fields can be any of the field strengths described throughout. For example, in some aspects, the field strength can be between 0.5 V/cm and 10 V/cm, 0.5 V/cm and 5 V/cm, 1 V/cm and 4 V/cm, or 0.5 V/cm and 2 V/cm. In some aspects, the field strength is about 0.5 V/cm, about 1 V/cm, about 2 V/cm, about 3 V/cm/or about 4 V/cm.

In some aspects, the subject does not have a tumor or cancer in the target site. In some aspects the subject does not have liver cancer. In some aspects, the subject does not have a tumor or cancer. In some aspects the subject has not been diagnosed with cancer, for example the subject has not been diagnosed with liver cancer, before applying the alternating electric fields. In some aspects, the subject has been diagnosed with diabetes but has not been diagnosed with cancer, for example liver cancer. In some aspects, the subject only has diabetes. In some aspects, the subject having diabetes has type 2 diabetes.

In some aspects, a magnetic field is not applied to the subject. Thus, in some aspects, only an electric field (e.g. alternating electric field) is applied to the subject.

### 1. Combination Treatment

Disclosed are one or more of the methods described throughout further comprising administering a diabetes therapeutic to the subject. Thus, an electric fields, such as an alternating electric field, and a known diabetes therapeutic can be used together to treat a subject with diabetes.

Disclosed are methods of treating a subject having diabetes comprising applying an alternating electric field to a target site of the subject for a period of time, the alternating electric field having a frequency and field strength, wherein FGF21 is increased in the subject, and further comprising administering a diabetes therapeutic to the subject. In some aspects the alternating electric field is applied in a single direction. In other aspects the alternating electric field is applied in multiple directions.

In some aspects, the diabetes therapeutic is administered prior to applying the alternating electric fields. In some aspects, the diabetes therapeutic can be administered minutes, hours, days, or weeks prior to applying the alternating electric fields. In some aspects, the diabetes therapeutic can be administered at least 1, 2, 3, 4, 5, 6, 7, 8, 9, 10, 11, 12, 13, 14, 15, 16, 17, 18, 19, 20, 21, 22, 23, or 24 hours prior to applying the alternating electric fields. In some aspects, the diabetes therapeutic can be administered at least 1, 2, 3, 4, 5, 6, or 7 days prior to applying the alternating electric fields. In some aspects, the diabetes therapeutic can be administered at least 1, 2, 3, or 4 weeks prior to applying the alternating electric fields.

In some aspects, the diabetes therapeutic is administered after applying the alternating electric fields. In some aspects, the diabetes therapeutic can be administered minutes, hours, days, or weeks after applying the alternating electric fields. In some aspects, the diabetes therapeutic can be administered at least 1, 2, 3, 4, 5, 6, 7, 8, 9, 10, 11, 12, 13, 14, 15, 16, 17, 18, 19, 20, 21, 22, 23, or 24 hours after applying the alternating electric fields. In some aspects, the diabetes therapeutic can be administered at least 1, 2, 3, 4, 5, 6, or 7 days after applying the alternating electric fields. In some aspects, the diabetes therapeutic can be administered at least 1, 2, 3, or 4 weeks after applying the alternating electric fields.

In some aspects, the diabetes therapeutic is administered simultaneously with applying the alternating electric fields. In some aspects, administering simultaneously includes administering a diabetes therapeutic while the subject is receiving alternating electric fields. In some aspects, administering simultaneously includes administering a diabetes therapeutic within seconds or minutes of applying the alternating electric fields.

Diabetes therapeutics are well known in the art. In some aspects, the diabetes therapeutic is metformin, Insulin therapy, a sulfonylurea, a non-SU secretagogues, an alpha-glucosidase inhibitor, a glinide, a thiazolidinedione, a GLP-1 receptor agonist, an SGLT2 inhibitor, a DPP-4 inhibitor, an incretin mimetic, an mylin analogue, a GIP analog, a peroxisome proliferator activated receptor or a dipeptidyl peptidase-4 inhibitor.

### D. Methods of Altering a Biological Pathway or Protein Expression

Disclosed are methods of increasing one or more of FGF21, blood glucose uptake, and brain-derived neurotrophic factor (BDNF) in a subject having diabetes comprising: applying an alternating electric field to a target site of the subject for a period of time, the alternating electric field having a frequency and field strength, wherein FGF21 is increased in the subject.

Disclosed are methods of increasing FGF21 in a subject having diabetes comprising: applying an alternating electric field to a target site of the subject for a period of time, the alternating electric field having a frequency and field strength, wherein FGF21 is increased in the subject.

Disclosed are methods of increasing blood glucose uptake in a subject having diabetes comprising: applying an alternating electric field to a target site of the subject for a period of time, the alternating electric field having a frequency and field strength, wherein FGF21 is increased in the subject.

Disclosed are methods of increasing BDNF in a subject having diabetes comprising: applying an alternating electric field to a target site of the subject for a period of time, the alternating electric field having a frequency and field strength, wherein FGF21 and BDNF are increased in the subject.

In some aspects, an increase of one or more of FGF21, blood glucose uptake, and BDNF in a subject having diabetes can be an increase compared to a subject that does not receive alternating electric fields. In some aspects, an increase of one or more of FGF21, blood glucose uptake, and BDNF in a subject having diabetes can be an increase compared to a subject that does not have diabetes but still receives the alternating electric field. In some aspects, an increase of FGF21 is at least a 2 fold increase in serum levels of FGF21.

In some aspects, the target site is a location on a subject to provide treatment with an alternating electric field. In some aspects, the target site comprises hepatocytes. For example, the target site can be the subject's liver. In some aspects, the target site is the subject's torso. In some aspects, the target site can be any area of the subject comprising organs or cells associated with the diabetes disease process.

In some aspects, the frequency of the alternating electric field can be any of the frequencies described throughout. For example, in some aspects, the frequency of the alternating electric fields is between 50 kHz and 10 MHz, 100 kHz and 1 MHz, 100 and 500 kHz, 150 and 500 kHz, or 500 kHz to 1 MHz. In some aspects, the frequency of the alternating electric fields is about 100, about 130, about 150, about 180, about 200, about 220, about 250, about 300, about 350, about 400, about 450, or about 500 kHz.

In some aspects, the field strength of the alternating electric fields can be any of the field strengths described throughout. For example, in some aspects, the field strength can be between 0.5 V/cm and 10 V/cm, 0.5 V/cm and 5 V/cm, 1 V/cm and 4 V/cm, or 0.5 V/cm and 2 V/cm. In some aspects, the field strength is about 0.5 V/cm, about 1 V/cm, about 2 V/cm, about 3 V/cm/or about 4 V/cm.

In some aspects, the subject does not have a tumor or cancer in the target site. In some aspects the subject does not have liver cancer. In some aspects, the subject does not have a tumor or cancer. In some aspects the subject has not been diagnosed with cancer, for example the subject has not been diagnosed with liver cancer, before applying the alternating electric fields. In some aspects, the subject has been diagnosed with diabetes but has not been diagnosed with cancer, for example liver cancer. In some aspects, the subject only has diabetes. In some aspects, the subject having diabetes has type 2 diabetes.

In some aspects, a magnetic field is not applied to the subject. Thus, in some aspects, only an electric field (e.g. alternating electric field) is applied to the subject.

### E. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for treating subjects with diabetes, the kit comprising equipment for applying alternating electric fields and a diabetes therapeutic. For example, the kits can comprise a pair of electrodes and a signal generator (e.g., a TTFields device such as Optune®, Novocure Ltd.) and one or more known diabetes therapeutics.

### Examples

FGF21 plays a major role in lowering blood glucose levels and could potentially be increased by application of alternating electric fields to treat type 2 diabetes.

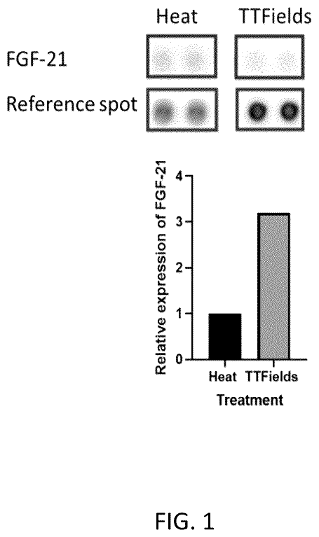

Cytokine array analysis of proteins secreted from rats that were applied alternating electric fields (150 kHz) for two weeks (to the torso) have shown that FGF-21 levels were increased following alternating electric fields application compared to heat sham treated rats in safety assays. Blood was drawn from rats prior to sacrifice and serum was produced. Serum was taken from the alternating electric fields treated and heat sham treated rats and cytokines were analyzed. These rats were not tumor baring indicating a direct involvement of alternating electric fields application on inducing secretion of FGF-21 levels in blood (FIG. 1).

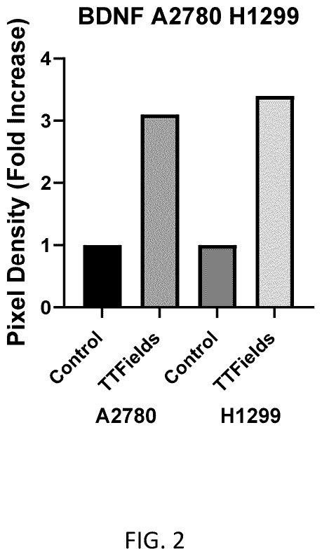

More supportive evidence was found in cytokine array analyses of proteins secreted from ovarian cancer cell line A2780 or Non-small cell lung carcinoma H1299 following TTFields application for 72 hours. Brain-derived neurotrophic factor (BNDF) levels are increased by 3.1 and 3.4 fold respectively in condition media derived from cell culture (FIG. 2). Increasing the levels of circulating BDNF has been shown to be useful in prevention and management of diabetes mellitus (Bathina et al., Brain-derived neurotrophic factor and its clinical implications. Arch Med Sci 2015 Dec. 10; 11(6):1164-1178).

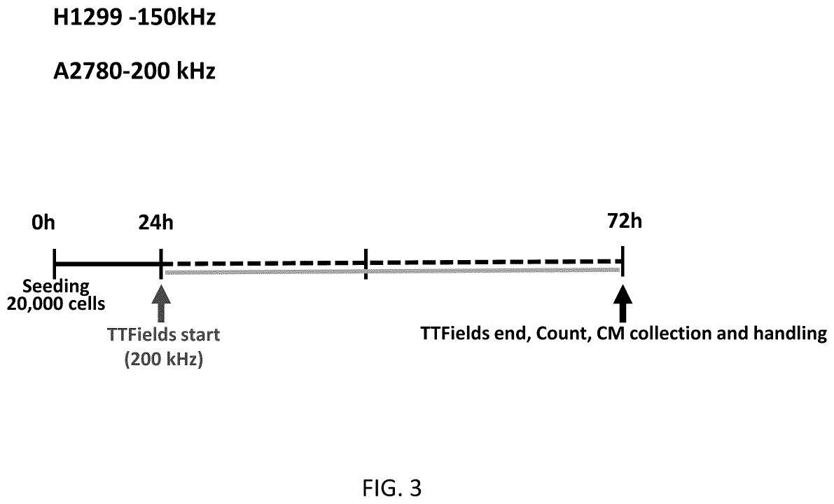

FIG. 3 shows the protocol used for FIG. 2. Volumes were evened using molecular Grade distilled water, CM was centrifuged and upper fluid was collected and kept in −20 degrees. Cell number was normalized after FACS count. Fold Change>3 was considered as up regulation following treatment with alternating electric fields.

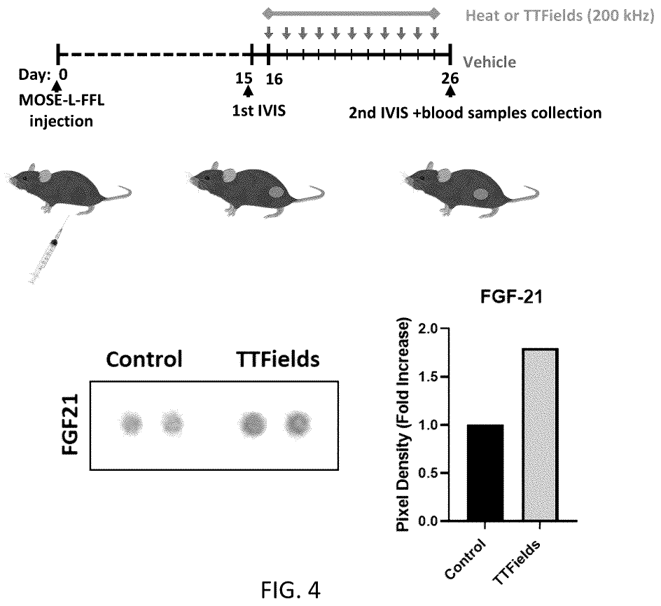

FIG. 4 shows that FGF-21 expression is upregulated following 10 days of alternating electric fields application in an ovarian cancer mice model. The mouse model is an orthotropic ovarian cancer mouse model. MOSE-L-ffl cancer 5000/5 μl cells were injected into 12 week old female mice. Mice were treated with alternating electric fields for 10 days using the INOVIVO system (Novocure, Haifa, Israel). Mice were sacrificed and blood samples were drawn and collected in designated blood serum tubes Centrifuged for 15 minutes in 1000 g and kept in −20° C. For the cytokine array kit analysis and quantification, the cytokine array kit (R&D systems ARY005B for human derived samples and ARY028 for mouse derived samples) was used to simultaneously detect 105 cytokines in each human cell line conditioned media (CM)/mice serum sample were collected according to the manufacturer's instructions. Array membranes were incubated for 1 hour at room temperature in blocking buffer (all reagents were supplied with the array kit). Prior to mix with reconstituted detection antibody cocktails, samples were incubated on the membranes overnight at 4° C. on a rocking platform shaker. All of the following steps were performed at room temperature, and all wash procedures involved three washes in 1× wash buffer for 10 minutes. After incubation with detection antibody, membranes were washed and incubated with streptavidin-conjugated horseradish peroxidase (1:2000) for 30 minutes on a rocking platform shaker. Unbound reagents were removed by washing, and the membranes were incubated in chemiluminescent detection reagent for 1 minute. To evaluate changes following alternating electric fields treatment vs. the control group spots were measured for pixel density using R&D systems quantification software (quick spots) and the results were analyzed as followed: [(protein pixel density in alternating electric fields treated sample)÷(protein pixel density in the control sample)].

Those skilled in the art will recognize, or be able to ascertain using no more than routine experimentation, many equivalents to the specific embodiments of the method and compositions described herein. Such equivalents are intended to be encompassed by the following claims.

We claim:

1. A method of treating a subject having diabetes comprising applying an alternating electric field to a target site of the subject for a period of time, the alternating electric field having a frequency and field strength,
wherein FGF21 is increased in the subject.

2. A method of treating a subject comprising: identifying a subject with diabetes, applying an alternating electric field to a target site of the subject for a period of time, the alternating electric field having a frequency and field strength,
wherein FGF21 is increased in the subject.

3. A method of treating a subject comprising: identifying a subject with diabetes, wherein the subject has a target site that is identified as being tumor-free, applying an alternating electric field to the target site of the subject for a period of time, the alternating electric field having a frequency and field strength,
wherein FGF21 is increased in the subject.

4. A method of treating a subject comprising: identifying a subject with diabetes, wherein the subject is also identified as being tumor-free, applying an alternating electric field to a target site of the subject for a period of time, the alternating electric field having a frequency and field strength,
wherein FGF21 is increased in the subject.

5. The method of any one of claims 1-4, wherein the target site comprises hepatocytes.

6. The method of any one of claims 1-4, wherein the target site is the subject's liver.

7. The method of any one of claims 1-4, wherein the target site is the subject's torso.

8. The method of any one of claims 1-7, further comprising administering a diabetes therapeutic to the subject.

9. The method of any one of claims 1-8, wherein the subject is not diagnosed with liver cancer.

10. The method of any one of claims 1-8, wherein the subject is not diagnosed with a tumor.

11. The method of any one of claims 8-10, wherein the diabetes therapeutic is administered prior to applying the alternating electric fields.

12. The method of any one of claims 8-10, wherein the diabetes therapeutic is administered after applying the alternating electric fields.

13. The method of any one of claims 8-10, wherein the diabetes therapeutic is administered simultaneously with applying the alternating electric fields.

14. The method of any one of claims 1-13, wherein the frequency of the alternating electric fields is between 50 kHz and 10 MHz.

15. The method of claim 14, wherein the frequency of the alternating electric fields is about 200 kHz.

16. The method of any one of claims 1-15, wherein the field strength is between 0.5 V/cm and 10 V/cm.

17. The method of claim 16, wherein the field strength is 1 V/cm.

18. The method of any one of claims 8-17, wherein the diabetes therapeutic is metformin, a sulfonylurea, a glinide, a thiazolidinedione, a GLP-1 receptor agonist, or an SGLT2 inhibitor.

19. The method of any one of claims 1-18, wherein the subject having diabetes has type 2 diabetes.

20. The methods of any of claims 1-19, wherein a magnetic field is not applied to the subject.

21. A method of increasing FGF21 in a subject having diabetes comprising: applying an alternating electric field to a target site of the subject for a period of time, the alternating electric field having a frequency and field strength,
wherein FGF21 is increased in the subject.

22. A method of increasing blood glucose uptake in a subject having diabetes comprising: applying an alternating electric field to a target site of the subject for a period of time, the alternating electric field having a frequency and field strength,
wherein FGF21 is increased in the subject.

23. A method of increasing brain-derived neurotrophic factor (BDNF) in a subject having diabetes comprising: applying an alternating electric field to a target site of the subject for a period of time, the alternating electric field having a frequency and field strength,
wherein FGF21 and BDNF are increased in the subject.

24. A method of treating a subject having diabetes comprising applying an alternating electric field to a target site of the subject for a period of time, the alternating electric field having a frequency and field strength,
wherein FGF21 is increased in the subject.

25. A method of treating a subject comprising: identifying a subject with diabetes, applying an alternating electric field to a target site of the subject for a period of time, the alternating electric field having a frequency and field strength,
wherein FGF21 is increased in the subject.

26. A method of treating a subject comprising: identifying a subject with diabetes, wherein the subject is also identified as being tumor-free, applying an alternating electric field to a target site of the subject for a period of time, the alternating electric field having a frequency and field strength,
wherein FGF21 is increased in the subject.

27. The methods of any of claims 24-26, wherein a magnetic field is not applied to the subject.

28. The method of any of claims 21-27, wherein the target site comprises hepatocytes.

29. The method of any one of claims 21-28, wherein the target site is the subject's liver.

30. The method of any one of claims 21-29, wherein the target site is the subject's torso.

31. The method of any one of claims 21-30, further comprising administering a diabetes therapeutic to the subject.

32. The method of any one of claims 21-31, wherein the subject is not diagnosed with a tumor of the liver.

33. The method of any one of claims 21-31, wherein the subject is not diagnosed with a tumor.

34. The method of any one of claims 31-33, wherein the diabetes therapeutic is administered prior to applying the alternating electric fields.

35. The method of any one of claims 31-33, wherein the diabetes therapeutic is administered after applying the alternating electric fields.

36. The method of any one of claims 21-33, wherein the diabetes therapeutic is administered simultaneously with applying the alternating electric fields.

37. The method of any one of claims 21-36, wherein the frequency of the alternating electric fields is between 50 kHz and 10 MHz.

38. The method of claim 37, wherein the frequency of the alternating electric fields is about 200 kHz.

39. The method of any one of claims 21-38, wherein the field strength is between 0.5 V/cm and 10 V/cm.

40. The method of claim 39, wherein the field strength is 1 V/cm.

41. The method of any one of claims 31-40, wherein the diabetes therapeutic is metformin, a sulfonylurea, a glinide, a thiazolidinedione, a GLP-1 receptor agonist, or an SGLT2 inhibitor.

42. The method of any one of claims 21-41, wherein the subject having diabetes has type 2 diabetes.

43. The methods of any of claims 21-42, wherein a magnetic field is not applied to the subject.
